(19) United States
(12) Patent Application Publication
(10) Pub. No.: US 2023/0250493 A1
(43) Pub. Date: Aug. 10, 2023

(54) KIT AND METHODS FOR CHARACTERIZING A VIRUS IN A SAMPLE

(71) Applicant: PERSEUS BIOMICS, Kortenaken (BE)

(72) Inventors: Volker LEEN, Kortenaken (BE); Theo LASSER, Echichens (CH); Johan HOFKENS, Huldenberg (BE); Jens H. GUNDLACH, Seattle, WA (US)

(21) Appl. No.: 18/012,879

(22) PCT Filed: Jun. 25, 2021
(86) PCT No.: PCT/IB2021/055694

§ 371 Date: Dec. 23, 2022

(30) Foreign Application Priority Data
Jun. 25, 2020 (WO) PCT/IB2020/056021

(51) Int. Cl. C12Q 1/70

(52) U.S. Cl. CPC C12Q 1/70

(57) ABSTRACT

The present disclosure concerns a method for detecting and characterizing a virus comprising the steps of providing a sample to analyze that is likely to contain a virus, extracting and preparing nucleic acids from the sample, sequence-specifically labeling the nucleic acid, e.g. by introducing fluorophores by contacting the nucleic acid with a methyltransferase or by introducing fluorescently labelled nucleotides after treatment with nickase, performing a genomic mapping analysis of the extracted nucleic acids and performing a computational analysis to detect the presence of and characterise at least one virus. The present disclosure also concerns a kit for carrying out the above method.

### FILED OF THE INVENTION

The present invention relates to the detection and characterization of pathogens in samples. More particularly, the present invention concerns a method for characterizing an infectious virus in a sample and a kit for carrying out the method.

### BACKGROUND

Viruses represent a significant portion of the high priority pathogens of concern to both human and animal health. The National Institute of Allergy and Infection Diseases′ (NIAID) prioritized listing of human pathogens includes the RNA viruses: Hanta viruses, Dengue, Ebola, Marburg, Lassa, Hepatitis A and C, West Nile, and a large number of encephalitis viruses. The USDA prioritized list of infectious animal diseases include four RNA viruses within their top six ranked pathogens: Foot and Mouth Disease Virus, Rift Valley Fever Virus, common swine fever virus, and Japanese encephalitis virus. A recent SARS-CoV-2 outbreak has reached pandemic levels and caused a global state of emergency. Influenza takes between 30000 to 80000 lives annually. Viral outbreaks of these pathogens have significant direct impact on human health or dramatic indirect impact by their disruption of food supplies and related economic considerations.

Effective and timely response to viral outbreaks is essential to track viral outbreaks. However, the current diagnostics are limited by both available technology and implementation. Hence, a fast, reliable and specific diagnostic method is needed.

Although human coronaviruses cause up to 30 percent of colds, they rarely cause lower respiratory tract disease. In contrast, animal coronaviruses are known to cause severe symptoms in animals. It has been speculated that the SARS virus originated in animals and mutated or recombined to permit it to infect humans. This theory is supported by preliminary evidence that suggests that antibodies to the isolates of the SARS virus are absent in those not infected with the virus. Recent studies suggest a pig origin.

SARS infections have been confirmed by detection of SARS RNA via PCR or via RT-PCR. PCR, while determining whether or not virus RNA is present in a sample, does not provide information as to whether a sample is infectious Also, stringent laboratory protocols need to be adhered to avoid cross contamination of samples. Whether a sample contains infectious virus can be determined by inoculating suitable cell cultures, such as Vero cells, with a patient specimen. Generally, such cell cultures are very demanding and require biosafety levels (BSL) 3 facilities.

Two detection methods for SARS targeting the presence of antibodies in the serum of a patient are enzyme linked immunoabsorbant assays (Elisa) and immunofluorescence assay (IFA). Here SARS infected cells are fixed to a microscope slide, and antibodies in a serum sample bind to viral antigen and are made visible by immunofluorescent labeled secondary antibodies against human IgM or IgG or both. Generally, IFA is performed by laboratories with BSL-3 facilities. Original antigen production for Elisa also often involves the use of SARS infected cells.

These situations demonstrate truly global threats with a tremendous impact on health care systems and far reaching negative consequences for national and global economics. The lack of fast, reliable and specific diagnostic methods and instruments is one of the main reasons that virus infections appear regularly and turn into pandemic global scenarios.

The highly contagious nature of SARS combined with an eminent mortality rate can be disruptive and costly in an increasingly globalized world. This was promptly recognized by the World Health Organization (WHO) in 2003 when the SARS epidemic spread beyond its place of origin in Guangdong Province, China, and a global alert was declared for the first time in WHO history. The outbreaks affected over 8,098 people and spread to more than 29 countries and regions in a short period of 6 months, with a mortality rate of up to 15%. Obviously, this disease is not a limited problem but one with profound consequences affecting sectors beyond public health care. As new cases appear to be re-emerging as of January 2004, the urgency remains for prompt identification and isolation of infected patients. Such need for recurrent screening and analysis is also observed in the 2020 COVID-19 Epidemic.

A novel coronavirus was identified as the etiological agent of SARS. Coronaviruses are enveloped viruses that contain a single-stranded, positive-sense RNA genome of 27.6 to 31 kb. Analysis of the nucleotide sequence of the novel SARS coronavirus (SARS CoV) showed that the viral genome is nearly 30 kb in length and contains 14 potential open reading frames.

However, detection technology, diagnosis, and treatment of the COVID-19 is subject to intense research efforts. Prompt identification and isolation of infected patients is not available and remains of paramount importance for disease control, since no drug or vaccine is presently available for this disease.

When SARS was first identified, disease management relied on travel and contact history and presentation of symptoms. Subsequently, with the identification of the SARS-CoV genome, several diagnostic tests based on the detection of viral RNA sequences by use of PCR have been designed and are now available. Based on these methods, the WHO has revised its criteria for case definition.

Despite the high sensitivity, these tests have inherent problems: it is unclear what types of samples (respiratory samples, saliva, stool, blood, or conjunctival fluid) from patients give the most reproducible RNA preparations; these RNA extraction protocols are not straightforward, and if not done well, may produce RNA preparations that are not useful for the reverse transcription step that converts viral RNA to DNA. The whole process of extraction, reverse transcription, and PCR can be time-consuming since confirmatory tests have to be done with several pairs of primers. False positives in the PCR amplification methods can arise, as was observed in August 2003 in Canada, when some patients infected with other human coronaviruses initially tested positive for SARS. Contamination in PCR laboratories is always a concern, leading to unnecessary quarantines.

To reduce the risk of contact with people who may have been exposed to the SARS causing virus, strict quarantine orders are served to those who have travelled to SARS-affected countries, those who had been in direct contact with SARS patients, and those with elevated temperatures. Early diagnoses of the disease during the early phase of infection could avoid unnecessary quarantines, reduce the stress to those concerned, and help doctors to decide on appropriate medical action and/or treatment. It is therefore vital to identify SARS patients as early as possible, with certainty and accuracy.

Animals also suffer from viral infections. Foot and mouth disease (FMD) is the most contagious transboundary animal disease affecting bovines and other cloven-hoofed animals. Significant economic losses result from its high morbidity, and from tourism and export trade restrictions imposed on affected countries. While the U.S. has not had a case of FMD since 1929, the country remains vigilant against its import and potential bioterrorism to protect against the devastating economic impacts of this disease. FMD is caused by infection of a picornavirus, a non-enveloped, positive-strand RNA virus. Previraemic infection is often localized to epithelial tissues of the nasopharynx, due to aerosolized airborne contamination. Active viral replication occurs during a preclinical phase of infection within this tissue and then continues in the lungs and endo-vascular system during the viremia phase. Viral replication of RNA virus is via an RNAdependent RNA polymerase, D3pol, encoded by the virus whereby the RNA genome of the virus is replicated and packaged within the cytoplasm of infected cells.

Current methodologies for assay of FMD rely on detection of viral antigens and/or viral RNA, and do not require these markers to be active nor intact. Both approaches typically require laboratory clinical analyses. While kits “ready to be used in the field” are becoming available for ELISA-based detection, they still suffer low sensitivity, and, most importantly, do not provide an indication of active/transmissible infection. Charleston et al. (Science 2011) indicate that the infectious period of FMD is much shorter than previously realized (mean 1.7 days) and animals are not infectious until approximately 0.5 days after the onset of clinical symptoms. As such, costly remediation measures of herd culling may be unnecessary if accurate, “in the field” methods for determination of infectiousness can be achieved.

The enumeration of all these scenarios has an important impact on health care systems and last but not least to the national economics, as long as reliable, fast and affordable diagnosis is missing.

The present invention overcomes the shortcomings of these assays and methods and provides a highly reliable, rapid diagnostic system for the detection of pathogenic viruses.

### SUMMARY OF THE INVENTION

To achieve the aforementioned objective, an improved system for viral profiling and/or profiling bacteria i.e. reading the genomic abundancy profile is provided based and organized by an interplay of microbiology concepts, novel RNA/DNA reader systems and algorithms.

To further achieve the aforementioned objectives, we describe a method for detecting the presence or absence of viruses while also providing the ability to distinguish between a multitude of different viruses.

An additional objective of this invention relies on methods related to microbiology, exploiting the reverse transcription of enzymes to translate the viral RNA code into its corresponding DNA sequence.

A further objective of this invention is to exploit the a priori known DNA signature of DNA strands to improve the identification accuracy of the DNA strand and in consequence the virus identification accuracy.

A further objective of this invention is the simultaneous detection of a whole variety of viruses.

An additional objective of this invention is to detect the viral insertion site in the host DNA strands.

Another objective of the invention is the detection and identification of viruses in a subject based on a single molecule level, without the need for a significant amplification step.

Additional advantages, objects and features of the invention will be set forth in part in the description and claims which follow and in part will become evident to those having ordinary skill in the art upon examination of the following or may be learned from practice of the invention. The objects and advantages of the invention may be realized and attained as particularly pointed out in the appended claims.

### BRIEF DESCRIPTION OF THE DRAWINGS

FIG. 1 is a flow-chart of a method of characterizing an infectious virus according to an example embodiment of the invention;

FIG. 2 depicts an example embodiment of the method for viral DNA detection;

FIG. 3 shows an intensity profile together with a site map of TGCA positions on the SARS-Cov-2 virus; and

FIGS. 4a and 4b shows a side to side comparison for the TGCA and the GTAC sites.

### DEFINITIONS, TERMS AND ELEMENTS

The following terms and related definitions are used in the present text.

“Subject” is used herein to mean any living being, human or animal. Nevertheless, the here disclosed method can be used for plants as well. As it is obvious for those skilled in the art, that subject in the context of this patent should mean any living body exposed to a viral infection.

“Sample” is used herein to mean first, any substance taken from a subject and undergoing a diagnosis based on the disclosed method. Secondly, our method applies equally well to any material like textiles, plastics, air filters, but not limited hereto. In summary, sample is used here for designating any living material and any solid or liquid or gaseous material exposed or invaded or containing a virus or viruses. A sample taken from a subject may contain biological material such as saliva, mucus, cheek swabs, nasal swabs, blood, fecal matter, urine, or substances from breather masks, dust recovered from air filters, surface swabs but not limited hereto. For efficient early detection in populations these samples may be pooled to determine the presence or absence of viral matter.

“Stretching” is used herein to mean depositing a DNA molecule onto a surface so that all vectors that point form a nucleotide n to the neighboring nucleotide n+1 or n-1 have a positive projection onto the vector from the first nucleotide to the last one. By these kinds of approaches, the base pair distance is increased and acts like an additional magnification for an optical reading. Effectively this means that a DNA forms a linear object, where the DNA strand along the stretching may have up to several micrometer, but in the lateral, perpendicular to the stretching direction is limited to several nanometers.

“Optical read out” is used herein to mean: a method that uses light signals to glean a specific information allowing the identification with high accuracy of viral species. Such signal or optical intensity profiles are put into relation with the genetic codes known and downloaded from a databank. A matching algorithm, as for example based on a cross-correlation or a neuronal network, but not limited hereto serves to relate with high accuracy the measured signal to a priori known RNA or DNA based information, allowing to assign the measured signal to a known genetic information.

cDNA: complementary DNA, cDNA is DNA complimentary to RNA. cDNA copies may be made using the enzyme reverse transcriptase (RT) or DNA polymerases having RT activity, which results in the production of single-stranded cDNA molecules. The single-stranded cDNAs may then be converted into a complete double-stranded DNA copy (i.e., a double-stranded cDNA) of the original RNA by the action of a DNA polymerase or a DNA accepting RT.

The term “substituted” as used herein refers to an organic group as defined herein or molecule in which one or more bonds to a hydrogen atom contained therein are replaced by one or more bonds to a non-hydrogen atom. The term “functional group” or “substituent” as used herein refers to a group that can be or is substituted onto a molecule, or onto an organic group. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents J that can be bonded to a substituted carbon (or other) atom include F, Cl, Br, I, OR′, OC(O)N(R′)2, CN, NO, NO2, ONO2, azido, CF3, OCF3, R′, O (oxo), S (thiono), C(O), S(O), methylenedioxy, ethylenedioxy, N(R′)2, SR′, SOR′, SO2R′, SO2N(R′)2, SO3R′, C(O)R′, C(O)C(O)R′, C(O)CH2C(O)R′, C(S)R′, C(O)OR′, OC(O)R′, C(O)N(R)2, OC(O)N(R′)2, C(S)N(R′)2, (CH2)0-2N(R′)C(O)R′, (CH2)0-2N(R′)N(R′)2, N(R′)N(R′)C(O)R′, N(R′)N(R′)C(O)OR′, N(R′)N(R′)CON(R)2, N(R′)SO2R′, N(R′)SO2N(R′)2, N(R′)C(O)OR′, N(R′)C(O)R′, N(R′)C(S)R′, N(R′)C(O)N(R′)2, N(R′)C(S)N(R′)2, N(COR′)COR′, N(OR′)R′, C(═NH)N(R′)2, C(O)N(OR′)R′, or C(═NOR′)R′ wherein R′ can be hydrogen or a carbon-based moiety, and wherein the carbon-based moiety can itself be further substituted; for example, wherein R′ can be hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or R′ can be independently mono- or multi-substituted with J; or wherein two R′ groups bonded to a nitrogen atom or to adjacent nitrogen atoms can together with the nitrogen atom or atoms form a heterocyclyl, which can be mono- or independently multi-substituted with J.

“Biorthogonal” is used herein to mean chemical reactions that can be used in biological systems, coupling one reactive group specifically with another reactive group: without side reactions; in neutral, aqueous solution; and under additional conditions that are compatible with the biological system.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

By the phrase “nucleic acid extraction reagent” is meant any reagent (e.g., solution) that can be used to obtain a nucleic acid (e.g., DNA) from biological materials such as cells, tissues, bodily fluids, microorganisms, etc. An extraction reagent can be, for example, a solution containing one or more of a detergent to disrupt cell and nuclear membranes, a proteolytic enzyme(s) to degrade proteins, an agent to inhibit nuclease activity, a buffering compound to maintain neutral pH, and chaotropic salts to facilitate disaggregation of molecular complexes. “Reactive group” refers to a chemical moiety capable of reacting with a partner chemical moiety to form a covalent linkage. A moiety may be considered a reactive group based on its high reactivity with a single partner-moiety, a set of partner-moieties, or based on its reactivity with many partners.

“DNA Mapping” refers to a process where sequence specific markers are introduced to a polynucleotide, and where the distance information between these markers yields information on the genetic makeup of the polynucleotide. DNA mapping may refer to all polynucleotides in a sample, including but not limited to genomic DNA, plasmid DNA, mRNA, tRNA and genomic RNA.

### DETAILED DESCRIPTION OF SEVERAL EMBODIMENTS

The disclosed method is used to test whether a subject is infected by, or has been exposed to a virus, wherein a sample from a subject is tested, and wherein specific viral DNA signature indicates that the subject is infected by or has been exposed to said virus.

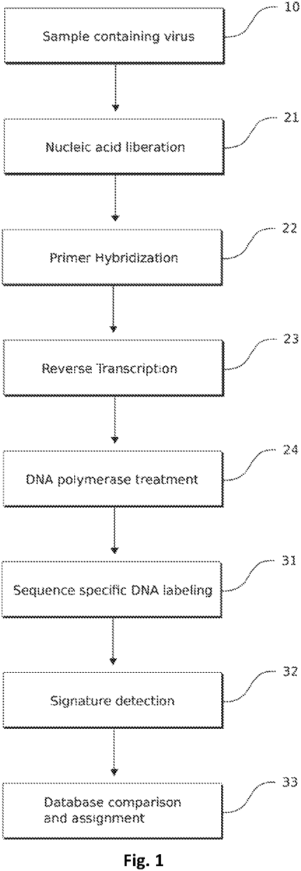

The disclosed method 100 is visualized in FIG. 1 and comprises 3 distinct steps, (10, 20, 30).

These steps can be subdivided as:
a first step of taking a sample 10 from a subject supposed to contain viruses;
a second step 20 of preparing the sample 10;
a third step of DNA mapping analysis 30 of the resulting DNA.

The step 20 of preparing the sample may comprise:

A nucleic acid extraction step 21, liberating the nucleic acid from the virus.

A hybridization step 22, which means the reaction with a primer and the viral RNA.

A reverse transcription reaction 23, where the RNA is processed by an inverse or reverse transcriptase, to produce cDNA from RNA virus.

A DNA polymerase reaction 24, together with one or a plurality of primers that are complimentary to the cDNA. The reaction results in double stranded DNA (dsDNA).

A reverse transcriptase walking on cDNA reaction 24, together with one or a plurality of primers that are complimentary to the cDNA. The reaction results in double stranded DNA (dsDNA).

The step of DNA mapping analysis 30 of the resulting DNA may comprise:

Labeling the dsDNA being 31 at a plurality of specific sites by labels suitable to generate a detectable signature.

Detecting the DNA signature off of separate single-molecules 32.

Using a software to compare the signature of single DNA molecules to a catalog of know signatures 33, thereby identifying the presence of a or a plurality of viruses.

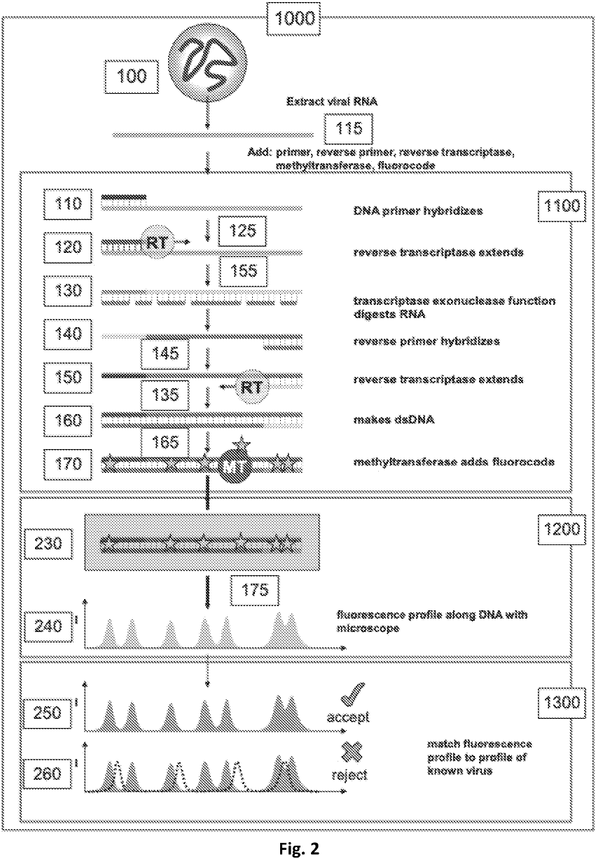

FIG. 2 describes an example of the invention for viral DNA detection through the disclosed method 1000, comprising 3 distinct workflow steps (1100, 1200, 1300).

Steps (1100, 1200, 1300) can be subdivided as:
step (1100) taking a sample (100) from a subject supposed to contain viruses.
Sequential or simultaneous addition of a primer
A reverse transcriptase, a methyltransferase and a fluorescently labeled cofactor (115).

A hybridization between the primer and a conjugated single stranded DNA (115) acts as an initiation of the reverse transcriptase, which will integrate and translate (125, 155) the viral RNA code into single stranded cDNA.

Optionally, a transcriptase-exonuclease will digest (155) the viral RNA.

The processing steps (140-160) are important for generating a double stranded DNA, carrying the full genome information of the viral RNA.

A reverse primer (150) will further interact with the reverse transcriptase (140, 145, 150, 135, 160, 165) to generate a double stranded DNA (dsDNA) molecule.

Finally, a methyltransferase enzyme integrates fluorophores at sequence-specific sites into the DNA strand.

Optionally, the method may further comprise a partial DNA amplification step. The amplification step may comprise a Polymerase Chain Reaction (PCR) with a low number of thermal cycles, for example between 1 to 5 thermal cycles. The amplification step enables to increase the sensibility of the method in order to detect and characterize very low level of viral genetic material in a subject.

The details of these steps are disclosed in the Pat. US 10,183,965 B2, this reference herewith incorporated by reference in its entirety.

As further disclosed in the Pat. US 10,183,965 B2 these DNA fragments may be stretched on a glass slide in order to read the specific signature i.e. an intensity profile of this DNA strand.

As indicated in the last part (1300) of this example workflow, this experimentally determined intensity profile is now compared with a priori known and calculated intensity profiles. The publicly available genomic information extends over a multitude of viruses/bacteria and is used for matching/comparing both profiles in order to use a statistical estimation for the best match between both profiles and such provide the knowledge about the presence/absence of bacteria and/or viruses.

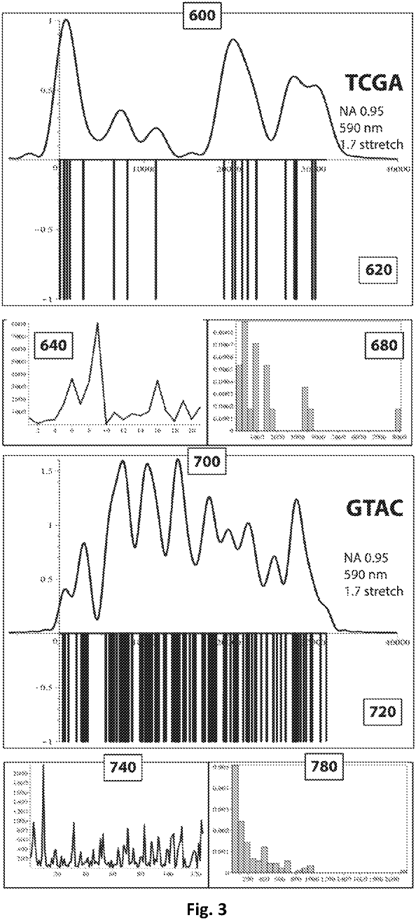

FIG. 3 shows an intensity profile (600) together with a site map of TGCA positions on the SARS- Cov-2 virus. In this given example the intensity profile is generated with a Rhodamine dye emitting at 590 nm and using a NA of 0.95 (numerical aperture). The peak intensities in 600 reflect well the density of the TGCA sites along the DNA strand. The small number of sites in this virus is indicated in the distance map (640) and further shown in the histogram 680.

The equivalent analysis for the GTAC sites changes due to the 5-fold higher number of GTCA sites (when compared to TCGA). The analysis of the GTCA sites is further detailed in the distance map (740) i.e. distance between neighboring sites and their distribution shown in the histogram (780).

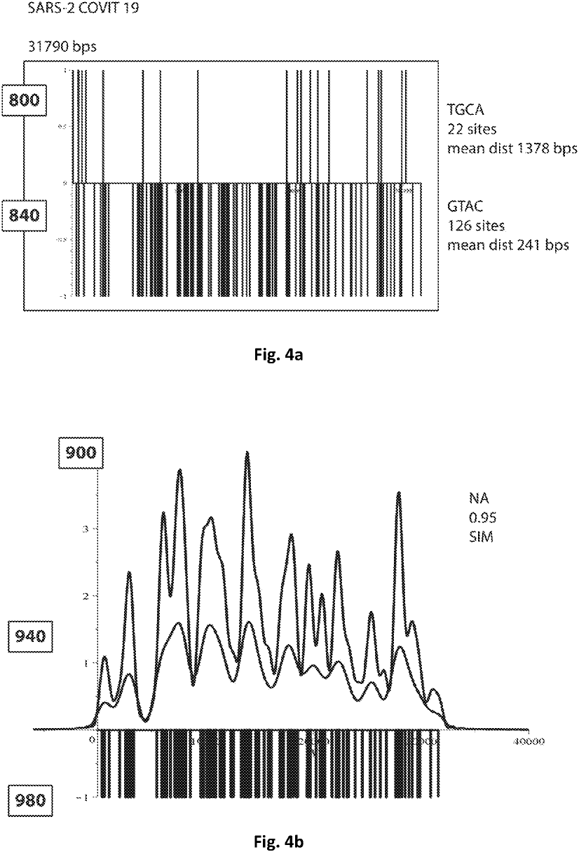

FIG. 4 shows the side to side comparison for the TGCA (800) and the GTAC (840) sites. Obviously, the density for the GTAC as already mentioned is much higher. The information content can be extracted with a much-increased contrast when using a super resolution technique like structural illumination microscopy (SIM) (but not limited hereto).

This is further demonstrated in (900, 940, 980) with a comparison of a classical imaging with an NA of 0.95 and a SIM imaging, where the increased resolution provides a more detailed view and finally a quality increase for the matching and recognition of the viruses.

In one embodiment, the type of viral is not known. In this case, the method is used to identify the type of virus from a catalog or databank of viruses.

In another embodiment, the method will indicate if the sample contains one or more viruses.

Should two or more viruses be identified in the sample the method will provide the relative abundance of the viruses. Depending on the sample prep the method can provide the absolute concentration of the viruses in the sample.

One embodiment of the present invention includes a step of preparing a reverse transcription reaction solution by mixing a sample that may contain an RNA virus and a thermostable reverse transcriptase. Here, the reverse transcription reaction solution refers to a solution containing a thermostable reverse transcriptase and other elements necessary for the reverse transcription reaction.

The thermostable reverse transcriptase used in the method of the present invention is not particularly limited to the present invention but is an enzyme that exhibits reverse transcription activity at 30° C. or higher, preferably an enzyme that exhibits reverse transcription activity at 50° C. or higher. More preferably, it refers to an enzyme that exhibits reverse transcription activity at 60° C. or higher, and more preferably an enzyme that exhibits reverse transcription activity at 65° C. or higher. For example, heat-resistant mutants of virus derived reverse transcriptases such as MMLV, AMV, Maxima, SuperScript I-IVHIV, RAV2, and EIAV, but not limited hereto.

Although the present invention is not particularly limited, examples of thermostable reverse transcriptase suitable used in step (23) of the present invention include reverse transcriptase derived from thermophilic bacteria and super thermostable bacteria, and DNAdependent DNA synthesis having reverse transcription activity. An active DNA polymerase can be preferably used. Although not particularly limited, for example, thermus thermophilus (Thermus thermophilus), thermus aquaticus (T. aquaticus), bacillus stearothermophilus (Bacillus stearothermophilus) (B. caldtenax (B.), And the like, and the trt gene product of Geobacillus stearothermophilus (Appl. Env. Microbiol. 70, p7140-7147 (2004), incorporated herein by reference.) or a variant thereof.

Elements necessary for the reverse transcription reaction include primers for cDNA synthesis having a sequence complementary to the RNA to be detected, salts, deoxyribonucleotides, and buffer components. These elements are added to a sample together with a thermostable reverse transcriptase and mixed to prepare a reverse transcription reaction solution. MgCI2 and KCI are used as the above-mentioned salts, but other salts may be added or changed to other salts as appropriate. The buffer component refers to a compound or mixture having an action of reducing fluctuations in the hydrogen ion concentration (pH) of the reaction solution. In general, a mixed solution of a weak acid and its salt or a weak base and its salt has a strong buffering action and is therefore widely used for the purpose of maintaining an appropriate pH. Various reaction buffers known in the field of biochemistry can be used in the present invention, and it is appropriate that they are set within a normal range in which reverse transcription reaction or nucleic acid amplification reaction is carried out.

In another aspect, the present invention provides polynucleotides, oligonucleotides or nucleic acids encoding or relating to a polypeptide of the invention or a biologically active portion thereof, including, for example, nucleic acid molecules sufficient for use as hybridization probes, PCR primers or sequencing primers for identifying, analyzing, mutating or amplifying the nucleic acids of the invention.

Primers for both RNA and DNA can be designed in line with current state of the art. (Li, Brownley, Primer design for RT-PCR, Methods Mol Biol. 2010;630:271-99, incorporated herein by reference) The functional equivalent of the nucleic acid comprises hybridization properties that are qualitative in nature and not necessarily identical in quantity to the nucleic acid (or a portion thereof). A portion of a nucleic acid of the invention comprises at least 15, preferably at least 20, more preferably at least 30 nucleotides. The nucleic acid sequence of the invention, or an equivalent thereof, has at least 80% homology, preferably at least 90% homology, more preferably at least 95% homology to the nucleic acid sequence of the invention or a portion thereof. More preferably, at least 99% homology.

The nucleic acid can be any length. The nucleic acid can be, for example, at least 6, 7, 8, 9, 10, 11, 12, 13, 14, 15,16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 100,110, 120, 125, 150, 175, 200, 250, 300, 350, 375, 400,425, 450,475 or 500 nucleotides in length. The nucleic acid can be, for example, less than 3, 4, 5,6, 7,8, 9,10, 11,12, 13,14, 15,16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 100, 110, 120, 125, 150, 175, 200, 250, 300, 350, 375, 400, 425, 450, 475, 500, 525, 550, 575, 600, 650, 700, 750, 800, 850, 900, 950, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500 or 10000 nucleotides in length. In a preferred embodiment, the nucleic acid has a length and a sequence suitable for detecting a mutation described herein, for example, as a probe or a primer.

In another embodiment, the present invention provides nucleic acid molecules that are suitable for use as primers or hybridization probes for the detection of nucleic acid sequences of the invention. A nucleic acid molecule of the invention can comprise only a portion of a nucleic acid sequence encoding a full-length polypeptide of the invention for example, a fragment that can be used as a probe or primer or a fragment encoding a biologically active portion of a polypeptide of the invention. The probe can comprise a labeled group attached thereto, e. g. a radioisotope, a fluorescent compound, an enzyme, or an enzyme cofactor. In various embodiments, the nucleic acid molecules of the invention can be modified at the base moiety, sugar moiety or phosphate backbone. Advantageously, incorporation of such probes into the dsDNA can aid in preselection of the polynucleotide signatures of interest.

In a specific embodiment, several of the steps of can be conducted in a single reaction vial. The method comprises a DNA mapping step (30), comprising a sequence specific DNA labeling 31. Among the methods suitable for sequence specific labeling of DNA (See, e.g. Zohar, Nanoscale. 2011, 3027-3039; Gottfried, Biochem. Soc. Trans., 2011,623-8 or WO2020005846, incorporated herein by reference) are enzymatic DNA labeling action, sequence specific hybridization of oligonucleotides or sequence specific DNA recognition by a molecule.

In a specific embodiment, the sequence specific DNA labeling action can be a DNA nicking enzyme which introduces a single strand nick into the DNA at a known location, followed by incorporation of fluorescent nucleotides by a polymerase enzyme (reference). In yet another embodiment, this sequence specific labeling action is a DNA methyltransferase that transfers a functional group to the DNA.

In a specific embodiment, the density of the labels transferred to the DNA is optimal for the recognition of the signature of single viral DNA molecules.

The DNA mapping analysis can be conducted along methods of the state of the art (see, e.g. Neely, Biopolymers. 2011, 298-311, Müller, Lab Chip, 2017,17, 579-590; WO2011150475A1, WO2014123822 and EP2859947, incorporated herein by reference).

In a preferred embodiment, the DNA mapping is optical DNA mapping.

Any RNA virus can be used as a detection target in the detection method of the present invention, and examples thereof include non-envelope RNA viruses. Examples of nonenveloped RNA viruses include viruses belonging to the Caliciviridae family (Norovirus (NoV), Sapovirus (SV), feline Calicivirus (FCV) etc.) and viruses belonging to the Reoviridae family (Rotavirus (Rota)), Examples include viruses belonging to the Picornaviridae family (echovirus (E), enterovirus (EV)), and the like. The feline calicivirus (FCV) listed above is an alternative virus that is widely used for evaluation of disinfectants and detergents in place of human norovirus (HuNoV) that cannot be easily cultured.

The method can be used to detect a wide variety of virus types, for example, any virus found in animals. In one embodiment of the invention, the virus includes viruses known to infect mammals, including dogs, cats, horses, sheep, cows etc. In a preferred embodiment, the virus is known to infect primates. In an even more preferred embodiment, the virus is known to infect humans.

Examples of human viruses include, but are not limited to, human immunodeficiency virus, herpes simplex virus, cytomegalovirus virus, varicella zoster virus, other human herpes viruses, caliciviridae virus, norovirus, respiratory syncytial virus, hepatitis A, B and C viruses, rhinovirus, human papilloma virus, influenza virus or corona virus. In a preferred embodiment of the invention, the virus is influenza virus or corona virus.

The method further comprises comparing the pattern of the labeled DNA to a pattern of labels on a database comprising reference RNA and DNA 33. It is understood to a person skilled in the art that this database can be a DNA database were viral RNA is listed as its corresponding DNA as exemplified in FIG. 2 (1300), an experimentally determined intensity profile can be compared with known and calculated intensity profiles. The publicly available genomic information extends over a multitude of viruses/bacteria and is used for matching/comparing both profiles in order to use a statistical estimation for the best match between both profiles and such provide the knowledge about the presence/absence of bacteria and/or viruses.

In a preferred embodiment, the method uses a software based algorithm to compare the measured signature of single DNA molecules to a catalog of know signatures (33), and by correlating the measured signature to the data based signature, providing a scoring criteria for identifying the presence of a or a plurality of viruses.

The detection method is either a qualitative or a quantitative method. When quantitative, the method allows for accurate determination of viral load and quantification of viral presence.

It is another object of the present invention to provide a kit for carrying out the method for characterizing a virus in a sample.

According to a specific embodiment of the invention, the kit comprises as selection of sample collection, nucleic acid extraction reagents, cleanup/concentration buffers and exchange/purification consumables, RT, library of primers, DNA polymerase and DNA reverse transcriptase. Furthermore, the kit may contain the reagents for the genomic mapping step.

The kit of the present invention prepares a reaction solution for successively sample collection, virus capsid disruption chemicals, chemicals making reverse transcription reaction solution containing viral sample, cDNA synthesis by a thermostable reverse transcriptase, dsDNA synthesis by a polymerase and sequence specific labeling of the resulting DNA. Ingredients necessary for this are contained as components.

Although not particularly limited, the kit of the present invention includes, for example, a buffer component, a thermostable reverse transcriptase, and deoxyribonucleotides such as dNTP and dUTP. Further, a surfactant, salts, a primer for reverse transcription reaction and the like may be included.

As another embodiment, for example, a kit containing a premix reaction solution containing a one-step buffer component, a thermostable reverse transcriptase, a thermostable DNA polymerase, a surfactant, salts, and deoxyribonucleotides such as dNTP and dUTP.

As a more preferred embodiment, in addition to the above-mentioned components, a reverse transcription reaction primer and at least one target nucleic acid amplification primer pair are contained, and an RTPCR reaction solution can be prepared simply by adding a sample. The primer can be a general primer or be designed for specific viruses or genetic elements.

The kit of the present invention may contain a thermostable enzyme having reverse transcriptase activity and DNA dependent DNA polymerase activity instead of the combination of thermostable reverse transcriptase and thermostable DNA polymerase. The reverse transcriptase can copy RNA and/or DNA.

Advantageously, the present invention enables direct detection of single molecule viral signatures in subjects. Through the unprecedented use of genomic mapping for RNA virus diagnostics, multiple virus species and their relative abundancies can be measured directly in a time and cost-effective manner.

By relying on a double viral recognition, both by the specific primer for reverse transcription and the subsequent analysis of the single molecule mapping signature, the accuracy and specificity of the method is of tremendous value in the avoidance of false positive and negative analyses.

Both RNA and DNA viruses can be measured in the same population, as the workflow generates a single read-out step but allows assignation versus database of each piece of DNA. This allows for accurate discrimination of virus and the detection of multiple viruses can be highlighted.

The invention additionally provides methods of determining the presence of SARS associated virus using the methods disclosed herein.

The invention also provides such methods for determining the presence and further including the step of reporting the result of said determination.

So far, the disclosed sample processing has been described for a single sample processing. It is obvious for those skilled in the art, that this sample processing can be easily conceived as a highly parallel workflow for an additional gain in processing time without compromising the accuracy of assignment of the measured signatures originating from the DNA fragments.

The mentioned examples are intended to illustrate but not to limit the present invention.

### EXAMPLES

The Full RNA sequence of SARS-C ov-2 length (29903 bases) was converted in-silico to dsDNA and labeled with methyltransferase MTaql (QUERY_STRING = « TCGA »). This provides 22 sequence specific labeling, at base pair locations 43, 493, 540, 822, 1165, 2637, 6039, 7527, 10717, 18305, 18311, 19205, 19523, 20315, 20945, 21896, 25155, 26138, 26355, 28114, 28474, 29753, with mean distance between base pairs 1414 base pairs STD 1709 base pairs. From genomic mapping, the resulting intensity profiles are provided in FIGS. 3 and 4b, highlighting the coverage and unique signature obtained from the described method.

So far, we have chosen the SARS-Cov-2 virus for explaining in detail our approach. However, our technique applies to any virus and/or bacteria where portions or the entirety of the RNA sequence.

### REFERENCES

Charleston B, Bankowski BM, Gubbins S, Chase-Topping ME, Schley D, Howey R, Barnett PV, Gibson D, Juleff ND, Woolhouse ME.., Science. Relationship between clinical signs and transmission of an infectious disease and the implications for control, 2011 Jun 10;332(6035):1263.

M. Kanehisa Nucleic Acids Res., use of statistical criteria for screening potential homologies in nucleic acid sequences, Nucleic Acids Res.; 1984, 12, 203-13.

Li K, Brownley A, Primer design for RT-PCR Methods Mol Biol. 2010;630:271-99.

Neely RK, Deen J, Hofkens J, Optical mapping of DNA: single-molecule-based methods for mapping genomes Biopolymers. 2011, 298-311.

Müller V, Westerlund F, Optical DNA mapping in nanofluidic devices: principles and applications. Lab Chip, 2017,17, 579-590.

PCR Technology, Principles and Applications for DNA Amplification, Henry A. Erlich, M stockton press New York.

WO2011150475

WO2014123822

EP2859947

Zohara H. and Muller S., Labeling DNA for single-molecule experiments: methods of labeling internal specific sequences on double-stranded DNA Nanoscale. 2011, 3027-3039.

Gottfried A, Weinhold E, Sequence-specific covalent labelling of DNA, Biochem. Soc. Trans., 2011,623-8.

WO2020005846

1. A method for detecting and characterizing a virus comprising the steps of:
a) Providing a sample to analyze that is likely to contain a virus;
b) Extracting and preparing nucleic acids from the sample;
c) Performing a genomic mapping analysis on the nucleic acids; and
d) Performing a computational analysis that includes a detection and/or a characterization the presence of at least one virus.

2. The method according to claim 1, wherein the sample is likely to contain an RNA virus and step b) comprises a reverse transcription reaction.

3. The method according to claim 1, wherein step b) further comprises a DNA polymerase reaction.

4. The method according to claim 1, wherein the genomic mapping step is an optical genomic mapping step.

5. The method according claim 1, wherein the reactions of steps (a) to (d) are carried out in the same container.

6. The method according to claim 1, further comprising modulating activity of a reverse transcriptase, polymerase, nickase or methyltransferase by adjusting the temperature, dNTP concentration, cofactor concentration, buffer concentration and any combination thereof during reaction.

7. The method according to claim 1, where the sample is obtained from an animal.

8. The method according to claim 1, where the sample is obtained from a human subject.

9. The method according to claim 1, where the sample is obtained from an environmental source.

10. The method according to claim 1, wherein the virus is chosen from influenza virus, parainfluenza virus, adenovirus, respiratory syncytial virus, metapneumovirus, togavirus, flavivirus, coronavirus or picornavirus.

11. The method according to claim 1, wherein multiple viruses are analyzed simultaneously.

12. The method according to claim 1, wherein the method further comprises a step of partial DNA amplification.

13. The method according to claim 12, wherein the partial DNA amplification step comprises a Polymerase Chain Reaction comprising between 1 to 5 thermal cycles.

14. A kit for carrying out the method according to claim 1, comprising at least a thermostable reverse transcriptase, deoxyribonucleotides, buffer components and components for sequence specific DNA labeling.
